(19) United States
(12) Patent Application Publication
(10) Pub. No.: US 2008/0249492 A1
(43) Pub. Date: Oct. 9, 2008

(54) Fiber material web

(75) Inventor: Andreas Schmidt, Duderstadt (DE)

Correspondence Address: GUDRUN E. HUCKETT DRAUDT, SCHUBERTSTR. 15A, WUPPERTAL, 42289, DE

(73) Assignee: McAirlaid's Vliesstoffe GmbH & Co. KG, Steinfurt (DE)

(21) Appl. No.: 12/062,537

(22) Filed: Apr. 4, 2008

(30) Foreign Application Priority Data
Apr. 5, 2007 (DE) 10 2007 016 959.2

(51) Int. Cl. A61F 13/15; D21H 27/00

(52) U.S. Cl. 604/374; 162/109

(57) ABSTRACT

A fiber material web of pulp fibers is made by pulp fibers being calendered and spot-connected or linearly connected without a binder in pressurized areas by fusion of the fiber bodies with generation of an embossed patter. The pulp fibers are chemically treated pulp fibers. The fiber material web is suitable for use in sanitary articles and as a filter material.

### BACKGROUND OF THE INVENTION

The invention concerns a fiber material web of pulp fibers in which the fibers are calendered and spot-connected or linearly connected in pressurized areas by fusion of the fiber bodies with generation of an embossed pattern. Moreover, the invention concerns the use of the fiber material web for producing sanitary articles as well as sanitary articles that contain the fiber material web as an absorbent core and the use of the fiber material web as filter materials.

In the field of hygiene, fiber material web of pulp fibers are widely used. The fiber material webs serve as absorbent cores for absorbing bodily liquids. They have the task of acquiring the bodily liquids quickly and absorb them permanently. A further property of a pulp web is the conveyance of liquid or air into structures positioned underneath. Since the fiber material webs often have direct body contact, they should be body-compatible as much as possible and should acquire the incoming liquid so that it is distributed well. Pulp has furthermore the advantage that it is biologically decomposable, i.e., it can be disposed of in appropriate landfills where it will rot completely without a trace. For their use in sanitary articles, the fiber material webs should be highly absorbent or have good conveying properties, should be soft and processible as a web. For increasing the absorption capacity, so-called super absorbers, i.e., polymers that will absorb water to form hydrogels, are often added to the fiber material matrix. The use of fiber material webs in the sanitary field makes it necessary that the materials used for the production of the sanitary articles contain additives and other auxiliaries only to a very limited extent. For processing the fiber material webs to absorbent cores, the fiber material webs are usually employed in the form of pulp nonwovens and airlaids. The connection of the individual pulp fibers to one another is achieved by binders.

The production of fiber material webs free of binders is also known. In Canadian patent application CA 2309998 A1, the disclosure of which is incorporated by reference herein, a method for producing, without employing additional binders, absorbent and coilable fiber material webs that are comprised of pulp fibers or pulp material or solid bleached sulphate board and that have a tear strength of at least 0.12 kN/m is disclosed. In the disclosed method a random pulp fiber layer is employed and pre-compressed at a relatively low pressure so that a loosely adhering nonwoven of minimal density and tear strength is produced. This nonwoven that contains moisture up to 5 percent by weight is introduced into the nip of a pair of calender rolls wherein a pattern of spot-shaped or line-shaped pressure areas is generated at a relative higher pressure so that the randomly positioned fibers are pressed against one another. The fibers are pressed against one another in the pair of calender rolls at a pressure of 250 to 600 MPa so that a non-detachable fusion of the fibers is realized and an embossed pattern is generated on the fiber material web. For producing the fiber material web according to the prior art, commercially available cellulose fibers are used that are known, for example, under the technical term fluff pulp. In this application, the cellulose fibers are stretched.

The use in the sanitary field requires that only minimal amounts of additives for increasing the absorption capacity are contained. U.S. Pat. No. 4,898,614, the disclosure of which is incorporated by reference herein, discloses fibers that are twisted and chemically reinforced. These fibers have a higher volume for acquiring liquid in comparison to stretched fibers. Processing of these fibers to sanitary articles however requires that a binder be employed. A sanitary article that contains these fibers is disclosed, for example, in U.S. Pat. No. 5,318,554 and U.S. Pat. No. 5,147,345. As a result of using the binder, part of the higher absorption rate resulting from the twisted structure is however lost again. Moreover, the binder is an additive that can impair the skin compatibility of the material in a sanitary article.

### SUMMARY OF THE INVENTION

The present invention has the task to provide a fiber material web that is suitable for use in sanitary articles and that has in comparison to prior art cellulose fibers an increased absorption rates. In case of a possible application as a filter material, the fiber material web should have a good conveyance rate and a minimal pressure drop. Moreover, for achieving these properties the use of chemical additives should be avoided entirely if possible because additives can cause incompatibility with the skin and because there is the risk that the additives will transfer onto the medium to be filtered in case of application as a filter.

Object of the present invention is a fiber material web of pulp fibers in which the fibers are calendered and spot-connected or linearly connected without a binder in pressurized areas by fusion of the fiber bodies with generation of an embossed pattern characterized in that chemically treated pulp fibers are employed.

Surprisingly, it was found that because of the changed structure of the pulp fibers the absorption rate and conveyance rate of the bodily liquid to be absorbed can be significantly increased. The bodily liquids are quickly acquired and conveyed, i.e., transported away, and absorbed (generally, curled or mercerized fibers absorb less than normal pulp). Also, it was founded in connection with super-absorbent polymers that, after the liquid has passed into deeper structures, the fiber web is significantly drier than a fiber web with untreated fibers. The fusion of the fiber bodies under pressure produces a spot-shaped or line-shaped connection between the fibers so that the fiber material web is imparted with a fixed structure without the volume of the pulp fibers being reduced significantly. It is not required to apply a binder or other additives to the pulp fibers in order to achieve the tear strength that is required for sanitary articles.

### BRIEF DESCRIPTION OF THE DRAWINGS

FIG. 1 shows schematically a cross-section of an absorbent pad with base layer and absorbent layer of pulp fibers schematically indicating the embossed pattern.

FIG. 2 shows schematically a fiber material web with reinforcement web.

FIG. 3 shows an absorbent pad where the base layer covers two edges of the absorbent layer.

### DESCRIPTION OF PREFERRED EMBODIMENTS

In the context of the present invention chemically treated pulp fibers are used. The chemical treatment can be, for example, curling or mercerizing. Curled fibers are twisted about their axis and/or coiled along an axis and they have a curled appearance. Especially preferred fibers have a curl index of at least 0.3, in particular of at least 0.5.

The curl index relates to a two-dimensional measurement of the curled fiber and is determined by viewing the fiber in a two-dimensional plane wherein the projected length L_{R} of the fiber is measured as the longest dimension of a rectangle that circumscribes the fiber and the actual length L_{A} of the fiber is measured. The curl index is accordingly calculated based on the following equation:

Curl Index=L_{R}/L_{A}−1

The values L_{R} and L_{A} are determined by image analysis. The determination method is known to a person skilled in the art and is inter alia disclosed in “Application of Image Analysis to Pulp Fibre Characterization: Part 1” by B. D. Jordan and D. H. Page, p. 104.114, Canadian Pulp and Paper Association (Montreal, Québec, Canada) as well as in the experimental part of European patent 0 429 112, the disclosure of which is incorporated by reference herein.

In a possible embodiment the curled fibers are chemically reinforced, i.e., the fibers are chemically treated in order to increase their strength and stiffness under dry and especially also under wet conditions. The strengthening of the pulp fibers can be realized intramolecularly and/or intermolecularly, i.e., within an individual pulp fiber or an individual cellulose molecule or between different molecules. In this way, the treatment with the reinforcement agent provides a twisted and/or coiled configuration. Suitable reinforcement agents for cellulose fibers are those that effect a chemical cross-linking by reaction of the hydroxyl groups in the cellulose molecules, for example, monomer cross-linking agents. Examples of monomer cross-linking agents are dialdehydes with 2-8 carbon atoms, monoaldehydes with 2-8 carbon atoms and a further acid group, or dicarboxylic acids with 2-10 carbon atoms. These compounds can react with at least two hydroxyl groups in the molecule of the cellulose chain or a neighboring chain in a fiber. Particularly suitable cross-linking agents are acetaldehyde, glyoxal, glutaraldehyde, glyoxalic acid, oxalic acid, malonic acid, succinic acid, glutaric acid, adipic acid, pimelinic acid, suberic acid, azelaic acid, sebacic acid, maleic acid and fumaric acid, malic acid, tartaric acid, -ketoglutaric acid, glutaric acid, glutamic acid, aspartic acid, oxosuccinic acid, as well as phthalic acid and isophthalic add. Polycarboxylates such as citric acid, tartrates, succinates etc. as disclosed in EP 0 429 112 are also suitable.

A further possibility of chemically treating cellulose fibers is mercerization. In this process, the cellulose fibers are treated with lye and optionally further additives so that intermolecular and intramolecular bonds in the cellulose molecule are broken and a decoiling effect results. Depending on the process conditions, i.e., whether the action of the lye is realized under tension or not, stretched fibers can also be obtained.

In a few fields of application it can be necessary that the fiber material web according to the invention has an increased tear strength. For these applications the fiber material web 2′ may contain a reinforcement web 4 (see FIG. 2). The latter can be, for example, placed between two partial fiber material webs 2a of pulp fibers and co-processed during the manufacture of the fiber material web 2′. The reinforcement web can be for example a fabric, a knit fabric, or a nonwoven web. Possible are also continuous films with perforations in the pressure areas which perforations are either torn or in the form of melted holes.

Depending on the application, the fiber material web according to the invention can also contain additional ingredients that are able to absorb or convey liquids, in particular, wound secretions and urine. In addition to the pulp fibers so-called super absorbers, odor-binding substances, fragrances, active carbon or components that bind other substances or materials that release substances, for example, inorganic adsorption agents such as zeolites as well as antibacterial substances and/or pigments, are also possible as ingredients.

The fiber material web according to the invention is suitable for producing sanitary articles such as patient pads, diapers, female sanitary napkins, incontinence products, inserts or pads for food packaging, filter material.

The fiber material web according to the invention is suitable as a conveyance layer for use in sanitary articles such as patient pads, diapers, female sanitary napkins, incontinence products, inserts for food packaging, filter material.

The fiber material web according to the invention is suitable in connection with super-absorbent polymers as a conveyance layer with significantly reduced moisture contents in the fibers or the fiber web after conveyance of liquid and after-drying by the super absorber in the fiber web for use in sanitary articles such as patient pads, diapers, female sanitary napkins, incontinence products, inserts/pads for food packaging, filter material.

A further object concerns accordingly the use of the fiber material web as described above in sanitary articles such as diapers, patient pads, female sanitary napkins, incontinence products, food packaging, filter material etc.

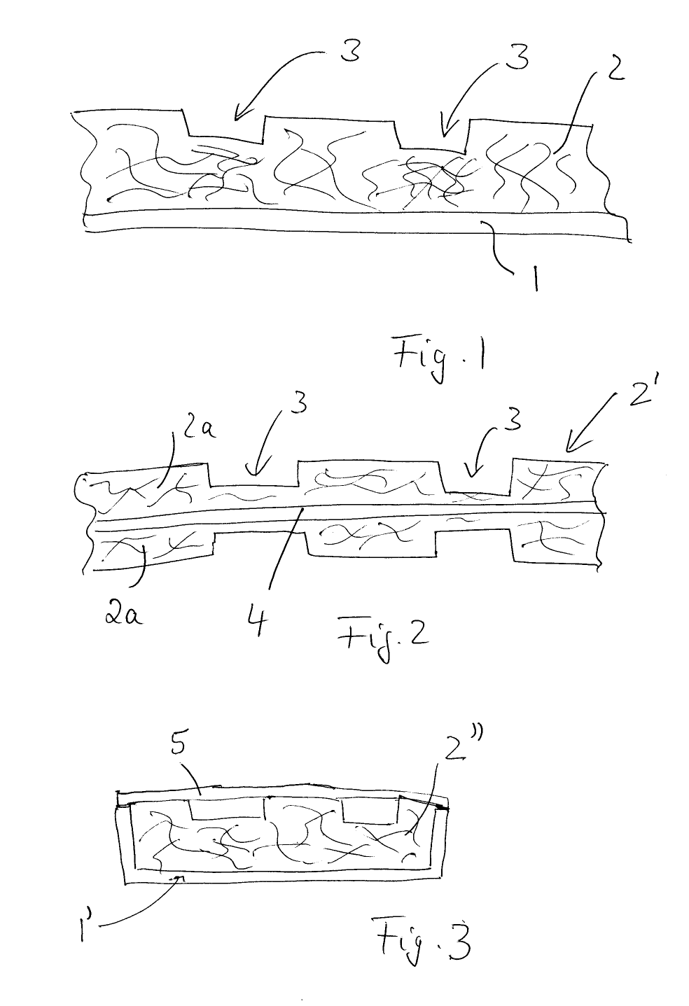

A further object of the present invention is a multi-layer absorbent insert or pad that is comprised of a base layer (1 in FIG. 1) that is impermeable for liquids and a layer of an absorbent material that is characterized in that the layer of an absorbent material (absorbent layer 2 in FIG. 1) is a fiber material web of chemically modified pulp fibers (schematically indicated in FIG. 1 by squiggles) and in that the fibers are calendered and spot-connected or linearly connected in pressurized areas by fusion of the fiber bodies with generation of an embossed pattern (3 in FIG. 1).

When used in a multi-layer absorbent pad, the fiber material according to the present invention can have different functions. In one possible embodiment, the absorbent pad has a layer of an absorbent material and a base layer that is impermeable for liquids. In this embodiment, the layer of absorbent material has the task to acquire the liquid to be absorbed, to distribute it so that the liquid is not only located within a certain area of the absorbent pad but is uniformly distributed, and to also retain the liquids, i.e., to store it. Retaining or storing of the liquid is designed to prevent that the absorbed liquid can escape again from the absorbent pad. For improving the storage capacity, it has been found to be advantageous when the layer of absorbent material contains additives such as super absorbers or inorganic absorption materials.

In a further possible embodiment, the absorbent pad has more than two layers, in particular a layer that acquires and distributes the liquid (acquisition and distribution layer) and a layer that stores the liquid. The fiber material web according to the invention in this embodiment represents the layer that acquires and distributes the liquid. It has the task to transport away the liquid as fast as possible from the object or body releasing the liquid and to distribute it across a large surface area within the absorbent pad. Storage of the liquid is realized in a layer positioned underneath that usually contains cellulose and similar materials in a reinforced form or as fluff pulp as well as adsorption agents and optionally additional ones of the aforementioned additives. Storage of the liquid can also be realized partially or entirety by means of absorbent material such as super absorbers within the fiber web in order to achieve a residual and/or after-drying of the fiber web. In connection with super absorbers, the volume of the fiber web will change additionally after the first liquid conveyance and after-drying of the fiber web. The liquid conveyance for liquid added later is thus increased. In comparison to the first liquid acquisition, the after-drying effect may be reduced for liquid added later.

In order to avoid direct contact of a body surface with the material described herein, the absorbent pad in an optional embodiment has a liquid-permeable cover layer 5 (FIG. 3) extending across the absorbent and liquid-distributing layer. It is particularly advantageous when this cover layer is comprised of a hydrophilic material. The hydrophilic material facilitates passage of aqueous liquids and thus the acquisition of this liquid by the absorbent material. The cover layer and/or the absorbent material can contain additionally antibacterial materials that prevent or minimize germ multiplication, for example, by vapor deposition of metals such as silver or aluminum.

In particular when the absorbent pad according to the invention is to be used in the field of hygiene, it is preferred when it is provided laterally with a drainage protection. For this purpose, in one possible embodiment the base layer 1′ is dimensioned somewhat greater than the absorbent layer 2″ so that the base layer 1′ can be folded about the edges of the absorbent layer and can thus enclose these edges so that acquired liquid cannot escape by way of the lateral edges (FIG. 3).

A further field of application of the fiber material web according to the invention is the use as a filter material for separating solid particles from a liquid or a gas.

A further object of the present invention is accordingly a filter material containing a fiber material web of pulp fibers in which the fibers are calendered and spot-connected or linearly connected without a binder in pressurized areas by fusion of the fiber bodies with generation of an embossed pattern characterized in that chemically treated pulp fibers are used.

The specification incorporates by reference the entire disclosure of German priority document 10 2007 016 959.2 having a filing date of Apr. 5, 2007.

While specific embodiments of the invention have been shown and described in detail to illustrate the inventive principles, it will be understood that the invention may be embodied otherwise without departing from such principles.

What is claimed is:

1. A fiber material web of pulp fibers in which the fibers are calendered and spot-connected or linearly connected without a binder in pressurized areas by fusion of the fiber bodies with generation of an embossed pattern, wherein the pulp fibers are chemically treated pulp fibers.

2. The fiber material web according to claim 1, wherein the pulp fibers are curled or mercerized.

3. The fiber material web according to claim 2, wherein the pulp fibers are curled and the curl index is at least 0.3.

4. The fiber material web according to claim 1, wherein the pulp fibers are chemically reinforced.

5. The fiber material web according to claim 4, wherein the pulp fibers are chemical reinforced by cross-linking.

6. The fiber material web according to claim 1, further comprising at least one additional ingredient selected from the group consisting of a super absorber, an odor-binding substance, a fragrance, active carbon, an inorganic absorption agent, an antibacterial substance and a pigment.

7. The fiber material web according to claim 6, wherein the inorganic absorption agent is a zeolite.

8. The fiber material web according to claim 1, further comprising a flexible reinforcement web.

9. An absorbent pad comprising:
a base layer impermeable for liquids;
an absorbent layer of an absorbent material comprised of pulp fibers in which the pulp fibers are calendered and spot-connected or linearly connected in pressurized areas by fusion of the fiber bodies with generation of an embossed pattern;
wherein the pulp fibers are twisted about their axis, coiled in a direction of their longitudinal axis; or twisted about their axis and coiled in the direction of their longitudinal axis.

10. The absorbent pad according to claim 9, wherein the base layer closes off at least two edges of the absorbent layer.

11. The absorbent pad according to claim 9, further comprising a cover layer arranged across the absorbent layer, wherein the cover layer is permeable for liquids.

12. The absorbent pad according to claim 8 in the form of a sanitary article.

13. The absorbent pad according to claim 12, wherein the sanitary article is a diaper, a patient pad, or a wound dressing.

14. The absorbent pad according to claim 8 in the form of a food packaging insert.

15. The absorbent pad according to claim 8 in the form of a filter medium.

16. A filter material comprising a fiber material web according to claim 1.
